(19) United States
(12) Patent Application Publication
(10) Pub. No.: US 2007/0225226 A1
(43) Pub. Date: Sep. 27, 2007

(54) C-REACTIVE PROTEIN APHERESIS

(75) Inventors: David J. Hammond, Laytonsville, MD (US); Julie Tait Lathrop, Falls Church, VA (US)

Correspondence Address: FOLEY AND LARDNER LLP;SUITE 500, 3000 K STREET NW, WASHINGTON, DC, 20007, US

(73) Assignee: The American National Red Cross

(21) Appl. No.: 11/690,318

(22) Filed: Mar. 23, 2007

Related U.S. Application Data
(60) Provisional application No. 60/785,359, filed on Mar. 24, 2006.

(51) Int. Cl. A61K 38/16; A61K 38/10; A61K 38/08; A61K 38/06; A61K 38/05; C07K 14/16; C07K 7/08; C07K 7/06

(52) U.S. Cl. 514/12; 514/13; 514/15; 514/14; 514/16; 514/17; 514/18; 530/324; 530/325; 530/326; 530/327; 530/328; 530/329; 530/330; 530/331

(57) ABSTRACT

The present invention provides ligands that can bind CRP with high affinity and high specificity. The present invention also provides a method of treating a condition of elevated CRP through apheresis, by reducing CRP level via its binding to a CRP-specific ligand ex vivo. Systems of performing apheresis to reduce CRP levels are also provided.

This application claims benefit to U.S. provisional patent application No. 60/785,359, filed Mar. 24, 2006 to Hammond et al., which is hereby incorporated by reference in its entirety.

### BACKGROUND OF THE INVENTION

The present invention relates to the treatment of conditions associated with sustained elevation of C-reactive protein (CRP). More specifically, the invention relates to using CRP-specific ligands to reduce CRP levels via ex vivo therapy.

Documents cited in this description are denoted numerically, in parenthetical, by reference to a bibliography below. Each of the references is specifically incorporated herein by reference.

CRP is a plasma protein, with a structure of five identical, non-covalently linked protomers, each of a molecular weight of 24-kDa, arranged as a pentameric ring structure with radial symmetry (6). Each protomer has two faces: a recognition face binding to phosphocholine and an effector face binding to C1q and Fcγ receptor. A variety of other known ligands bind to CRP, such as phophoethanolamine, chromatin, histones, fibronectin, small nuclear ribonucleoproteins, laminins, and polycations.

CRP is an acute phase protein secreted by hepatocytes, which increases dramatically, from its normal level of <1 mg/L to 100-1000 mg/L within hours, in response to infection or injury (1). Since the expression of CRP is not influenced by age or pharmacotherapy, it is considered a reliable marker for tissue destruction, necrosis, and atherosclerosis. Its measurement is widely used to monitor various inflammatory states, angina pectoris, end-stage renal disease, rheumatoid arthritis and atherosclerosis (see 20 for references).

In physiological terms, CRP has both pro- and anti-inflammatory effects (2). CRP expression is up-regulated at the transcriptional level by the cytokine interleukin-6 (IL-6), and its expression can be further enhanced by interleukin-10 (IL-1β). On the other hand, CRP has a stimulation effect on IL-6 expression, which generates a positive feedback cycle.

In addition to a role for CRP as a non-specific indicator of inflammation (3), the literature has associated elevation of CRP level with other conditions, such as cardiovascular disease, metabolic syndrome, and colon cancer (4, 5), an up-regulation of expression of adhesion molecules in endothelial cells, an increase of low density lipoprotein (LDL) uptake into macrophages, and inhibition of endothelial nitric-oxide synthase expression.

Increasingly, elevated CRP appears to be a mediator of diseases or conditions detrimental to human health, including but not limited to: cardiovascular disease, hypersensitivity complications of infections, e.g., rheumatoid fever and erythema nodosum leprosum; inflammatory disease, illustrated by rheumatoid arthritis, juvenile chronic arthritis, ankylosing spondylitis, psoriatic arthritis, systemic vasculitis, polymyalgia rheumatica, Reiter's disease, Crohn's disease, and familial Mediterranean fever; allograft rejection, as may occur in renal transplantation; malignancy, such as lymphoma and sarcoma; necrosis associated, for instance, with myocardial infarction, tumor embolism, or acute pancreatitis; and trauma, such as that occasioned by a burn or a fracture.

In a recent model of cardiovascular disease, a specific inhibitor of CRP, 1,6-bis(phosphocholine)-hexane, was found to abrogate increase in infarct size and cardiac dysfunction produced by human CRP injected into rats undergoing acute myocardial infarction (24). This further confirms CRP's importance in the treatment of cardiovascular disease.

Accordingly, there is a need for a methodology to modulate CRP levels without impacting the ability of a patient to elicit an appropriate acute phase reaction.

Apheresis is a procedure to deplete a component selectively from a patient's blood ex vivo and to return the treated blood into circulation of the patient. This procedure has proved useful in removing LDL, antibodies, inhibitors of clotting factors, and other pathophysiological agents (14-15). All conventional LDL-cholesterol reduction strategies (21) have practical limitations, however, to the extent that they are apheresis platform-specific, retain a large extracorporeal volume during processing, which limits use with pediatric patients, and/or are labor intensive.

Removal of LDL also has a proven effectiveness in decreasing CRP levels, albeit modestly (10-13). The procedures do not remove CRP specifically. Rather, they affect CRP bound to LDL only, not free CRP. Thus, many patients with an elevated level of CRP and a normal level of LDL will not benefit from these procedures or from another, high-dose statin treatment, which also reduces LDL and, indirectly, CRP (7-9).

The rationale for therapeutic apheresis is reviewed by McLeod (14). One impediment to current procedures in this context is the lack of highly specific ligands for the target pathogenic proteins, which restricts the use of apheresis to depletion of abundant proteins. CRP typically is present in trace amounts, and an apheresis technique has yet to be proposed that targets the specific removal of CRP. A suitable ligand would have to display both high specificity and very high affinity for CRP, thereby to accommodate the shortcomings of apheresis and also compete with natural CRP ligands in the blood. Recently, about 500,000 candidates from a small molecule library were evaluated by Pepys et al. (24) for inhibitors to CRP. None were found. Consequently, the investigators embarked upon a synthetic program based on the crystal structure of CRP-phosphocholine complex before synthesizing 1,6-bis-phosphocholine) heptane as an inhibitor for CRP. These investigators did not suggest that the ligand may be useful for removal of CRP from whole blood nor was it evaluated as such. Thus there remains a need to identify ligands useful for therapeutic apheresis of CRP.

Furthermore, CRP is reported to have a half-life of less than one day. Thus, the resultant expectation that circulating CRP would be quickly replaced de novo militates against its candidacy for any direct aphereis procedure.

### SUMMARY OF THE INVENTION

Accordingly, the present invention provides an approach for treating, through apheresis, conditions that are associated with a sustained elevation of CRP in vivo. In particular, an apheresis method for treating a subject with a condition of sustained elevation of CRP comprises, pursuant to the invention, (A) providing a support upon which is immobilized a ligand that has high affinity and high specificity for CRP, such that a CRP binding element is formed, and (B) bringing the element into ex vivo contact with body fluid from the subject, whereby CRP level in the subject is reduced. The subject can be any subject that produces CRP, including mammalian subjects, such as humans.

In accordance with another aspect of the invention, an apparatus for CRP apheresis, comprised of such a CRP binding element, is incorporated into an apheresis system, useful for treating the aforementioned diseases and conditions. Thus, such a system of the invention comprises (A) a support upon which is immobilized a ligand that has high affinity and high specificity for CRP, such that a CRP binding element is formed, and (B) apparatus for bringing the support into contact ex vivo with bodily fluid, such as whole blood or plasma, from a subject, thereby to affect CRP level in the bodily fluid, and for returning the bodily fluid, depleted of CRP in this fashion, to the subject.

In yet another embodiment, the invention provides a ligand that has high affinity and high specificity for CRP, comprising a peptide, wherein the association constant between said CRP and said ligand is at least 10⁶M.

### BRIEF DESCRIPTION OF THE FIGURE

FIG. 1 shows the results of an assay to determine whether the specified ligands bind CRP. The results show that three of the tested ligands bound CRP under the testing conditions.

### DETAILED DESCRIPTION

The present inventor has discovered that CRP ligands are accessible for selection, based on the above-mentioned criteria of specificity and affinity, and that apheresis therapy therefore can targeted to CRP per se, in both free and bound form. The phrase “bound CRP” denotes CRP that is coupled with one or more other agents. In some embodiments, these ligands bind CRP non-covalently.

The identification of suitable, CRP-specific ligands, as described in detail below, is remarkable in several aspects. First, an ability to target bound CRP provides valuable data on the role of CRP and its binding partners. Importantly, a ligand that binds CRP and inhibits the interaction of CRP with its physiological binding partners, due to competition for a specific binding site, is particularly useful in drug development. Second, a specific interaction between an immobilized ligand and CRP, pursuant to the present invention, allows for identifying, quantifying, and/or selectively removing CRP-ligand complex from blood, plasma, plasma derivatives, and other biological samples.

Accordingly, the present invention provides for selectively decreasing the circulating concentration of CRP by means of an apheresis system that includes a CRP capture device comprised of a CRP-specific ligand. This approach does not affect the ability of the body to produce a significant level of CRP as an acute phase reactant. The invention improves the prognosis, for example, for cardiovascular disease in patients with both elevated and normal levels of LDL.

In accordance with the present invention, apheretic removal of CRP is accomplished by use of a CRP-specific ligand, and resultant CRP reduction may be combined with the action of other mediators as well. For instance, a CRP-specific ligand may be used to remove CRP selectively from blood, via apheresis according to the invention, in combination with a conventional measure taken to reduce LDL, too. Conventional measures include pharmaceuticals, such as statins. Such a combination apheresis system may have additive or potentiating effects by removing two harmful mediators of cardiovascular disease. In another instance, a CRP-specific ligand may be used for apheresis, pursuant to the invention, in combination with a ligand that targets IL-6, thereby to interrupt the cycle of CRP stimulation of IL-6 and vice versa.

Reduction of CRP by its specific binding to a chosen ligand, pursuant to the invention, has significance as well in the context of a condition of below-average cholesterol and elevated CRP. About 30% of fatal heart attacks occur in people with “normal” cholesterol levels. Such patients may have an underlying inflammatory condition, so breaking the inflammatory positive feedback loop may improve the treatment of patients with chronic inflammation. Chronic inflammation may be caused by an autoimmune disease, although a significant number of older patients have chronic inflammation of unclear origin.

As indicated above, the present invention contemplates that a CRP ligand that is suitable for apheresis is selected based on these criteria: (1) the ligand must have high affinity for CRP, preferably with an association constant of >10⁶M, which can be measured using well-known conventional techniques, such as those described in (25); (2) the ligand must be able to bind CRP in both free and bound forms. Preferably, the ligand is selected as well for a fast association rate, in order that the apheresis procedure is performed effectively within a reasonable time period.

The employment of CRP apheresis will decrease the level of CRP from ≧3 mg/L to a normal level of <2 mg/L and preferably less than 1 mg/L. Factors such as (i) blood volume circulating through the apheresis device and (ii) the rate of CRP synthesis versus the rate of CRP removal would be expected to influence the amount of CRP reduction and, hence, determine the frequency of apheresis.

I. Identification of a CRP Ligand

A key aspect of the invention relates to identification of a suitable CRP apheresis ligand, which is achieved, according to the invention, through screening a combinatorial library of synthetic peptides for a ligand that binds to CRP with appropriate affinity and specificity in the presence of blood or plasma. Preferably, the ligand does not co-purify (deplete) other important plasma proteins, does not activate platelets, factor VII, complement or the angiotensin-converting enzyme, and does not bind significant amounts of other plasma proteins.

To these ends, therefore, a ligand with an association constant of above 10⁶ is preferred. A ligand with a weaker association may bind CRP with sufficient avidity by virtue, for instance, of its multimeric interactions with each protomer of CRP pentamer.

To select such a ligand, a screening program pursuant to the invention would involve identification of a variety of ligands that bind generally to CRP, using, for example, the Bead Blot described below. These ligands could be produced at 1 gram scale, and their affinities and capacities for CRP then would be determined by standard techniques, such as equilibrium isotherm analysis (22). Their specificities also would be determined, e.g., by identification of other proteins bound to the affinity resins, by standard methods such as gel electrophoresis and mass spectrometry. Again through the use of conventional techniques, the ligands also would be evaluated for their impact on blood chemistry, including activation of plasma proteins (coagulation factors, fibrinolysis, complement), red blood cell physiology (hemolysis, and survival and half-life), and platelet activation and survival. According to the data thus generated would a candidate ligand or ligands be selected, from those initially identified.

In one embodiment of the invention, the synthetic peptide of the combinatorial library comprises 2′-naphthylalanine, in addition to natural amino acids, but does not contain Met or Cys. In another embodiment, the synthetic peptide does not contain Met, Cys or Gln at the N-terminal position. In yet another embodiment, the synthetic peptide comprises amino acids in L-form, except for the N-terminal residue, which can be in either L- or D-form.

A number of different libraries can be screened, in keeping with the present invention, and the selected ligands are between 3 to 25 amino acids in length or between 3 to 15 amino acids in length, for example. Thus, the ligands can be 3, 15, or 25 amino acids in length. In some embodiments, the ligands are 4-6 amino acids in length. According to one embodiment, the D-form residue in the N-terminus of the ligand is preferred, since it provides stability against digestion by exo-peptidases present in plasma. In another embodiment, the ligand does not comprise chemically unstable amino acids, such as tryptophan, cysteine, and methionine.

In addition, a ligand for use in the invention preferably exhibits both biological and chemical stability, particularly but not exclusively against enzymatic digestion in the blood. To this end, the peptide structure of selected ligands may be modified to generate analogs that have essentially the same binding characteristics as the identified ligands but that are synthesized onto a different scaffold or are synthesized from different monomers.

Thus, the peptide backbone may be replaced by a triazine, to provide greater chemical stability to alkali and sterilization conditions, as well as potential resistance to digestion by proteases present in the blood that contacts the device. Optimization of selected ligands also can be addressed through retro-inverse modifications, which yield an analog with a sequence and a chirality that is inverted, relative to the normative structure, which may confer resistance to proteases or improve the specificity of the ligand. Additional improvement to ligand specificity may be achieved by systematic point mutation of the amino acids in the ligand. Furthermore, optimization of ligand density should maximize the specificity of the ligand for CRP and reduce the cost of the ligand.

Screening a peptide library for ligand having high affinity and specificity for CRP is preferably accomplished using a Bead Blot technology described by Hammond et al. (16) and (28), the contents of which are hereby incorporated by reference in their entirety. Briefly, this technology entails synthesizing or immobilizing on a chromatography resin support a library of affinity ligands, which may be composed of several types of monomers, including amino acids. Such “peptide ligands,” generally from 1 to 10 amino acids in length, preferably are synthesized on chromatography beads via the split, couple, and recombine combinatorial approach (23,27). The resultant combinatorial bead library is incubated with CRP-containing human plasma or whole blood, to allow for ligand binding of the CRP, and non-bound protein is removed by washing.

The beads that bind CRP are selected by the following method. Briefly, the library is incubated with a starting material that contains CRP. All proteins in the mixture, including CRP, will bind to their corresponding ligands through affinity interactions.

After incubation and washing, 10 μl of the loaded libraries are mixed with 990 μl 0.5% low melting point agarose and poured on top of a 10 ml, 1.0% agarose gel. The gel is placed on a wick extending into a tank of transfer buffer. A protein-binding membrane is placed on top of the gel, facing the beads, so that the bound proteins are transferred overnight by capillary action with transfer buffer and captured on the membrane. During transfer, the transfer buffer permeates through the gel and the membrane and in the process dissociates bound protein from the beads according to the strength of the affinity interaction and the composition of the transfer buffer. A variety of transfer conditions and transfer buffers may be used. To transfer protein from a high affinity ligand, one employs strong chaotrope, such as 6M guanidine.

Upon removal of the membrane from the gel, the location of beads that had bound CRP from loaded library is determined by detecting the presence of CRP using anti-human CRP antibody, such as C6 monoclonal antibody, product of Abeam plc of Cambridge, UK, and a monoclonal antibody marketed by Hytest Ltd. of Turku, Finland (catalog number 4C28WB-0.5, BioDesign). The antibodies may be used in a conjugated form or may be coupled to a detection system, using a secondary antibody or streptavidin phosphatase, for instance. This produces a film with spots indicating the position of detected protein(s). The film and the gel are superimposed and the spots aligned with beads. White beads associated with spots indicate potential CRP ligands. These beads are selected, and the ligands thereon are sequenced. A resin bearing the ligand is synthesized at gram scale.

Bead resins that efficiently bind CRP are further evaluated in terms of robustness, reproducibility, affinity, capacity for CRP, and potential for interference with blood components. In addition, the resins are assessed for binding proteins other than CRP, using analytical methods such as SDS-PAGE and multi-dimensional protein identification.

II. Choice of Support for a CRP Ligand

To facilitate binding and separation of CRP-ligand complex in a sample, the ligand preferably is attached to an inert support, such as a membrane or resin.

Exemplary supports are: naturally occurring polymer, such as cross-linked albumin; polysaccharides, such as agarose, alginate, carrageenan, chitin, cellulose, dextran and starch; synthetic polymers such as polyacrylate, polyhydroxy methacrylate, polystyrene, polyacrolein, polyvinylalcohol, polymethacrylate, polyester, hyperfluorocarbon; inorganic compounds such as glass, silica, kieselguhr, zirconia, alumina, iron oxide and other metallic oxides. An insoluble support can be subjected to cross-linking or other treatments to increase physical or chemical stability, and can be formed into various shapes, including but not limited to fibers, sheets, rods, beads, and membranes.

An inert support may require the introduction of reactive groups such as amines, epoxy groups, and the like, for the subsequent covalent attachment of the ligand. This can be achieved by any of a number of conventional techniques, such as using plasma (electrical gas discharges, frequently in the radio wave and microwave frequency range) for surface activation and modification of a polymer. Thus, argon plasma treatment in the presence of oxygen will create peroxides on the polymer. An alternative approach entails grafting of charged molecules, using low-temperature plasma treatment.

III. Coupling CRP Ligand Onto a Support

In a preferred embodiment, a CRP-specific ligand is immobilized on the support via interaction between the ligand and any of a variety of reactive chemical groups presented by the support. These groups may be incorporated in the polymer during polymerization of the polymer or may be introduced by post-manufacture treatment, including plasma treatment, as described above.

Illustrative of these groups, available commercially for the direct coupling to the ligand, are CNBr, epoxy, and 2,2,2-trifluoroethanesulfonyl chloride (tresyl) groups. These are available through Tosoh Bioscience of Montgomeryville, Pa., Bio-Rad of Hercules, Calif., and GE Healthcare of Uppsala, Sweden. A resin that is tresyl- or epoxy-functionalized, for example, can cross-link with the ligand via an amino or sulfhydryl group. Alternatively, ligands can be coupled to an appropriate acceptor such as carboxy, amino, and formyl derivatives, through the use of homobifunctional cross-linkers, e.g., glutaraldeyde, sulfosuccinimidyl-4-(N-maleimidomethyl) cyclohexane-1-carboxylate, and 1-ethyl-3-[3-diaminopropyl]carbodiimide hydrochloride. These chemicals covalently attach a specific group on the ligand, frequently an amine, with a similar group (i.e., amine) on the support. Although coupling can be performed in a single reaction, these cross-linkers are relatively inefficient due to cross-linking ligands to other ligands. Heterobifunctional cross-linking reagents, such as N-hydroxysuccinimide, imidoester and EDC, react with two different groups, e.g., amine-carboxy, amine-sulfhydryl, and sulfhydryl-hydroxyl. Thus, ligands may be engineered with a single group and cross-linked to a resin through a second specific group.

The coupling strategy may be incorporated into ligand design to maximize coupling efficiency. For instance, the ligands may be synthesized, using solid-phase synthesis with a single C-terminal sulfhydryl or carboxy group, to facilitate coupling to the resin. Solid-phase synthesis may be performed using aminomethyl polystyrene macroporous, p-methyl BHA resin (Applied BioSystems, Inc. of Foster City, Calif.), Rink Amide resin, or Wang resins illustrated by Novabiochem® products of EMD Biosciences, Inc, an affiliate of Merck KGaA (Darmstadt, Germany), inter alia. An alternative strategy for generating an affinity resin entails incorporating the ligand into the manufacture of the resin, during the polymerization process.

In practice, a spacer such as a polyethylene oxide polymer or an amino acid, e.g., β-alanine and ε-amino caproic acid, is inserted between the coupled ligand and the support.

IV. Manufacturing the CRP Binding Element Comprised of the CRP Ligand and the Support

A CRP binding element may be assembled by placing the immobilized ligand-support matrix, as described above, into a cartridge that, in its simplest form, is a chromatography column. Such columns are commercially available, e.g., from Bio-Rad and GE Healthcare. The column may be of a conventional cartridge design, fluidized bed, expandable bed, or a monolith. Ideally, the size of the resin beads of the column would be sufficient to let blood cells pass through the columns, i.e., with an average diameter in the order of 60 μm or above.

Alternatively, the ligand support may be available in a membrane format and incorporated into the manufacture of filters that are used in the blood processing industry. Such filters are available from MacoPharma, Pall Corporation, Millipore, Terumo, Fresenius, and Asahi, among others. In some cases the solution containing the biological target agent, CRP, also will contain larger entities such as red blood cells, leukocytes and aggregates of various sizes. It is desirable to allow the large aggregates to flow through the solid matrix or support without interfering with the ability of the target biological agent to bind to the support.

This requires pore spaces in the solid matrix that are large enough to accommodate the flow of the large entities without adversely affecting their function. It is preferred, in other words, that the pore size is such as to allow passage of cells like red blood cells, without damage or clogging. In other circumstances, it is desirable to filter the large particles to facilitate adsorptive separation of the smaller target agents. Non-woven fiber or web, also referred to as melt blown polymer fiber or spun-bound web, is well known in the art and is used for filtration and separation of particles (17, 18).

A methodology is available for fabricating particle-impregnated, non-woven fiber (19) that could be used in an apheresis protocol according to the present invention. By this method, functionalized particles (see illustrative chemistries, supra) are blown into the polymer fibers during the melt blowing stage in production. More specifically, the non-woven fiber filter material is produced by dispersing, in a medium, a mass of a great number of small fiber pieces, each having a fiber diameter of not more than 10 mm and a length of about 1 cm, together with spinnable and weavable short fibers having an average length of 3-15 mm.

Another design of the CRP binding element, according to the invention, involves combining CRP reduction with LDL reduction. A design along these lines could employ a device with two compartments: a larger compartment, preferably containing a porous matrix characterized by pore sizes larger than 10 μm, and a plurality of resins in the matrix, for the binding not only of LDL but also of CRP associated with LDL; and a second compartment, e.g., with a membrane that carries ligand to CRP, to bind CRP that is not LDL-associated.

The assembled CRP binding element would be made sterile, e.g., by exposure to radiation or steam, in a conventional manner.

V. Integrating the CRP Binding Element Into an Apheresis System

The CRP binding element may be integrated into any apheresis system substituting a CRP binding element, as described, for the corresponding part(s) associated with binding/removal of the target agent. The resulting CRP apheresis system then can be employed for CRP reduction, according to the invention.

Thus, the CRP binding element could be substituted in apparatus with a design along the lines of the Adacolumn, a single-use adsorptive apheresis device, which is connected to a blood pump with flow rate detector, pressure monitor and air sensor, or the Adacircuit infusion line system (15). In this design, two 16-18 gauge needles are inserted into vascular access sites, such as bilateral antecubital fossa veins. The element is primed with saline optionally containing heparin. The priming fluid is collected in a waste container after the procedure begins. The patient's whole blood is continuously drawn into the line circuit using a blood pump and heparin is added into the line blood prior to entering the device. Treated whole blood then is returned to the patient, via the contralateral vascular access, and no replacement fluid is required. A typical procedure likely would take about sixty minutes.

An alternative design, the MATISSE system, includes the Fresenius Hemoadsorption Machine 44008 ADS with the Fresenius MATISSE EN 500, both products of Fresenius HemoCare Absorber Technology GmbH of Bad Homburg, Germany. The MATISSE EN 500 contains macroporous beads immobilized with human serum albumin (HSA) to bind endotoxins. Pursuant to the invention, those beads could be replaced, in terms of design modification, by the affinity adsorbent for CRP-apheresis therapy as described above. To that end, dual vascular access (16-18 gauge) probably would be required to process approximately 1.5 blood volumes in a period of over four hours. The patient's own blood would be anti-coagulated continuously with citrate, prior to passing through the absorber element of the invention. On the assumption that all CRP was present only in the accessible vasculature, about two-thirds of the vascular CRP would be expected to be depleted for each blood volume processed, pursuant to this embodiment of the invention.

A further design variation, according to the invention, would be a departure from that of the LIPOSORBER LDL adsorption device. The latter employs two LDL adsorption columns in parallel, one or both of which would be replaced, in design terms, by a CRP binding element of the invention. Thus, the patient's blood would be withdrawn via a venous access and enters the plasma separator. As blood flowed through the hollow fibers of the plasma separator, the plasma would be separated and pumped into one of two adsorption elements. As the plasma passed through the element, the target agent would be adsorbed selectively, and the plasma thus depleted would exit the column, for recombination with the blood cells exiting the separator, all of which would be returned to the patient via a second venous access. When the first element was loaded, a computer-regulated machine could switch the plasma to the second element, if necessary. The plasma remaining in the first element would be returned to the patient, and the element then would be regenerated, eluting the target agent(s), CRP and possibly LDL, into the waste lines. After elution the column would be re-primed, ready for the next cycle of adsorption, allowing for continuous treatment. A typical treatment would last 2-4 hours and probably would have to be repeated every two to three weeks.

The detailed description of the present invention continues by reference to the following example, which is illustrative only and not limiting of the invention.

### EXAMPLE 1

### Synthesis of Ligands

Libraries were constructed based on the methods of (26, 27, 28). They were synthesized as hexamer peptide ligands with a spacer by Peptides International (Louisville, Ky.) on Toyopearl AF Amino 650M resin (Tosoh Biosciences, Montgomeryville, Pa.). The ligands were linked to the base resin via a spacer and were synthesized according to the split-couple-recombine method, using all of the natural amino acids with the exception of methionine (Met), which tends to oxidize, and cysteine (Cys), which forms disulfide bonds within and between ligands. Glutamine (Gln), which tends to circularize following deamination, was omitted from the amino terminal position. 2′naphthylalanine, a stable tryptophan analog, as an additional source of aromatic diversity. D-amino acids, which are less sensitive to exopeptidase activity than the L-isomers and may also inhibit endopeptidase activity, were used at the amino terminal position, while L isomers were used at all of the internal positions. In the text and table, small letters denote D-isomers of amino acids, capital letters denote L-isomers, and Z denotes 2′naphthylalanine.

Specific ligands were scaled up by direct synthesis of each ligand on gram quantities of the original resin, using the same chemistry as for library synthesis, but with the defined, appropriate amino acids incorporated at each position.

### EXAMPLE 2

### Selection of CRP Apheresis Ligands

Peptide ligand libraries were synthesized on Toyopearl AF amino 650 M or AF Epoxy 650 M resins, as described in Example 1, with the first 5 positions synthesized comprising equal amounts of the natural L-amino acids with the exception of Met, and Cys and with addition of 2′-naphthylalanine. The N-terminal position included D as well as L-isomers, but excluded the inclusion of Gln. The “epoxy” library was synthesized with a cysteine derivative linking the synthesized ligand to the epoxy group via a thio-ether bond through the sulfhydryl group of the cysteine. (Each bead has multiple copies of a single ligand, and different beads will have different ligands.)

A library of hexamer peptide ligands, synthesized on Toyopearl 650 M amino library (Tosoh Biosciences, Montgomeryville, Pa.) by Peptides International, Louisville, Ky., was swollen and equilibrated in CPD solution (citrate, phosphate dextrose solution; product of Macopharma of Lille, France) diluted 1:7 in phosphate buffered saline, pH 7.4 (150 mM NaCl, 10 mM phosphate). 500 μl aliquots of swollen, equilibrated library were dispensed into 10 ml Polyprep chromatography columns (Bio-Rad, Hercules, Calif.). Human CRP (Novagen, San Diego, Calif.) was spiked into 5 ml citrated whole blood or plasma to a final concentration of 100 ng/ml or 50 ng/ml, respectively. The CRP-spiked blood or plasma was incubated with the equilibrated library for 1 hour at room temperature, with rotation. Plasma proteins, including CRP, will bind to their corresponding ligands through affinity interactions to resins.

After incubation, the unbound fraction was drained by gravity and the column was washed with 5 ml diluted CPD plus 0.05% Tween-20 (Sigma-Aldrich, St Louis, Mo.), followed by 2×5 ml diluted CPD. This produced the washed “loaded” library.

Bead blots were prepared by adding 10 μl of the blood or plasma loaded libraries containing approximately 25,000 beads, along with 2-3 μl of alignment beads, to 990 μl 0.5% low melting point agarose. Each mixture was poured on top of a 10 ml, 1.0% agarose gel (Pierce).

Alignment beads were used to improve identification and selection of CRP binding beads. Protein G sepharose beads were non-covalently bound with mouse IgG. This was detected by subsequent incubation with alkaline-phosphatase-labeled goat anti-mouse IgG (Pierce Biotechnology, Rockford, Ill.). The alignment beads generated a signal by forming a red precipitate on the beads upon incubation with chromogenic alkaline phosphatase substrate Fast-Red (Sigma-Aldrich, St. Louis, Mo.).

The gel was placed on a wick extending into a tank of transfer buffer. A PVDF membrane was placed on top of the gel, facing the beads, so that the bound proteins were transferred overnight by capillary action with transfer buffer and captured on the membrane. During transfer, the transfer buffer permeates through the gel and the membrane and in the process dissociates bound protein from the beads according to the strength of the affinity interaction and the composition of the transfer buffer. A variety of transfer conditions and transfer buffers may be used. To transfer high affinity ligand, strong chaotrope, such as 6M guanidine, was employed.

Upon removal of the membrane from the gel, the location of beads that had bound either mouse IgG from alignment beads or human CRP from loaded library was determined. CRP was detected by using mouse anti-human CRP antibody (Sigma-Aldrich, St Louis, Mo.), followed by alkaline phosphatase labeled goat anti-mouse IgG secondary antibody (Pierce Biotechnology, Rockford, Ill.), which detected the presence of CRP-derived and alignment bead-derived antibodies. This produced a film with spots indicating the position of detected protein(s). The film and the gel were superimposed and the spots aligned with beads, the majority of which were red alignment beads. White beads associated with spots indicated potential CRP ligands. These beads were selected, and their ability to bind CRP was confirmed by re-equilibrating and re-incubating the beads with CRP in blood or plasma.

The experiments were repeated twice. Two beads from the spiked blood and six beads from the spiked plasma, respectively, were selected. Ligand sequencing was performed by automated Edman degradation using a Procise 494 protein sequencer, product of Applied Biosystems.

The sequences derived from the beads from the spiked whole blood were: Leu-Gly-Thr-Tyr-Ile-Ala (SEQ ID NO: 1) and Gly-Asn-Gln-Lys-Trp-Gly (SEQ ID NO: 2), respectively. The sequences derived from the beads from spiked plasma were: Glu-Ser-Phe-Ala-Nal-Nal (SEQ ID NO; 3), Val-Leu-Arg-Pro-Trp-Lys (SEQ ID NO; 4), Val-Glu-Nal-Asn-Asn-Asn (SEQ ID NO: 5), Lys-Nal-Pro-Asp-Leu-His (SEQ ID NO: 6), Trp-Nal-Gln-Lys-Asn-His (SEQ ID NO: 7), His-Gly-Tyr-Ile-Gly-Leu (SEQ ID NO: 8), where Nal represents 2′-naphthylalanine. The ligands were all D at the amino terminus.

### EXAMPLE 3

### Measurement of Ability of Resins to Bind CRP

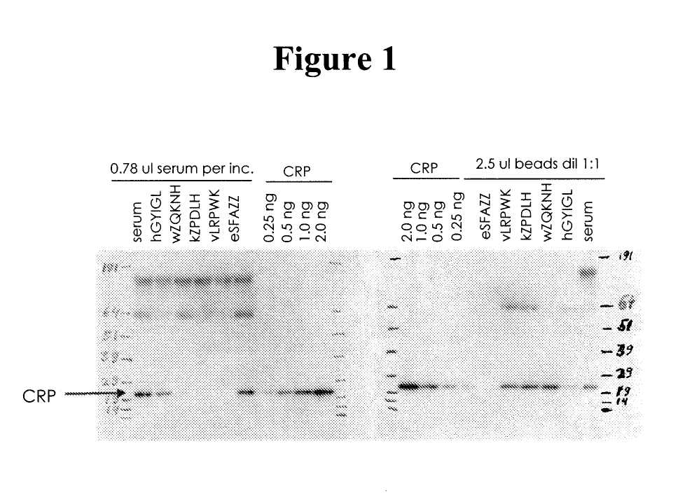

Several of the sequences identified in Example 2 were synthesized at gram scale to assay their ability to bind CRP. 90 μl of resin of ligands eSFAZZ (SEQ ID NO: 3), hGYIGL (SEQ ID NO: 8), vLRPWK (SEQ ID NO: 4), wZQKNH (SEQ ID NO: 7) and kZPDLH (SEQ ID NO: 6) (Z denotes 2′naphthylananine, small letters denote D amino acids, and capital letters denote L amino acids) were incubated with 300 μl of serum containing endogenous CRP, for two hours at room temperature. The supernatant was collected and the protein bound to the beads was eluted by incubation at 70° C. for ten minutes in 2×LDS sample buffer (Invitrogen). 10 μl of incubation supernatant or resin eluate was loaded per lane in an LDS gel and the proteins transferred to a nitrocellulose membrane. CRP was detected on the membrane using mouse anti-human CRP monoclonal antibody CRP-8 (Cat#C-1688, Lot# 094K4803), C=9.1 mg/ml (Sigma-Aldrich, St. Louis, Mo.) and affinity purified phosphatase labeled goat anti mouse IgG(□) human serum adsorbed (Cat#075-1802, Lot# XE084) (KPL, Gaithersburg, Md., USA). The results are shown in FIG. 1.

No CRP was detected in the supernatants of vLRPWK (SEQ ID NO: 4), WZQKNH (SEQ ID NO: 7), and kZPDLH (SEQ ID NO: 6), as shown in FIG. 1, indicating that these resins had bound all available endogenous CRP. This was confirmed by the detection of CRP in the eluates from these resins. The opposite is true for the remaining resins. Thus, vLRPWK (SEQ ID NO: 4), wZQKNH (SEQ ID NO: 7), and kZPDLH (SEQ ID NO: 6) appear to bind endogenous CRP under these conditions. It should be noted that all three of these ligands have a positively charged amino acid at the carboxy terminus (K or H) while none of the negative resins have the positively charged residue.

The equilibrium capacity of wZQKNH (SEQ ID NO: 7) for binding CRP was determined with CRP spiked into 200 μl of plasma or citrate buffer. 80 μl of wZQKNH resin was mixed with the spiked materials and allowed to incubate for two hours at room temperature. The supernatant was collected and CRP remaining in the supernatant (unbound fraction) was measured by ELISA. The apparent dissociation constant (the reciprocal of the association constant) for CRP bound from buffer was 12.8×10⁻⁹ M whereas the apparent dissociation constant for CRP bound from plasma was 370×10⁻⁹M, indicating a level of interference in plasma, possibly arising from CRP associating with other plasma proteins which may alter its dissociation constant.

### CITED PUBLICATIONS

Each of the following publications and each publication cited above is incorporated herein by reference, in its entirety.
1. M. B. Pepys, M. L. and Baltz 1983, Acute phase proteins with special reference to C-reactive protein and related proteins (pentaxins) and serum amyloid protein, Adv. Immunol. 34, 141-212.
2. M. B. Pepys and G. M. Hirschfield 2003, C-reactive protein: a critical update, J. Clin. Investigation 111, 1805-1812.
3. P. M. Ridker, 2004, High-sensitivity C-reactive protein, inflammation, and cardiovascular risk: From concept to clinical practice to clinical benefit, American Heart J. 148, 519-526.
4. P. M. Ridker, N. Rifai, L. Rose, J. E. Buring, N. R. and Cook 2002, Comparison of C-reactive protein and low-density lipoprotein cholesterol levels in the prediction of first cardiovascular events, N. Engl. J. Med., 347, 1557-1565.
5. H. Sun, T. Koike, T. Ichikawa, K. Hatakeyama, et al. 2005, C-Reactive protein in atherosclerotic plaque, American Journal of Pathology 167, 1139-1148.
6. S. Black, I. Kushner and D. Samols 2004, Minireview C-reactive protein, Journal of Biological Chemistry 279, 48487-48490.
7. P. M. Ridker, C. P. Cannon, D. Morrow et al. 2005, C-Reactive protein levels and outcomes after statin therapy, N. Engl. J Med. 352, 20-28.
8. S. E. Nisses, E. M. Tuzcu, P. Schoenhagen 2005, N. Engl. J. Med. 2005, 29-38.
9. M. Aikawa and P. Libby, 2004, Expert Viewpoints —Atherosclerotic inflammation: the final frontier? Can. J Cardiol, 20, 631-634.
10. P M Moriarty, C. A. Gibson, J. Shih and M. S. Matias 2001, C-reactive protein and other markers of inflammation among poatients undergoing HELP LDL apheresis, Atherosclerosis 158, 495-498.
11. J. Kobayashi, S. Katsube, M. Shimoda, K. Furuhashi et al. 2002, Single LDL apheresis improves serum remnant-like particle-cholesterol, C-reactive protein, and malondialdehyde-modified-low-density lipoprotein concentrations in Japanese hypercholesterolemic subjects, Clin. Chim Acta 321, 107-112.
12. C. Otto, H. C. Geiss, K. Empen, and K. G. Parhofer 2004, Long-term reduction of C-reactive protein concentration by regular LDL apheresis, Atherosclerosis, 174, 151-156.
13. H. Mabuchi, T. Higashikata and M-a Kawashiri 2004, Clinical applications of long-term LDL-apheresis on and beyond refractory hypercholesterolemia, Transfusion and Apheresis Science, 30, 233-243.
14. B. McLeod 2005, Transfusion and apheresis: Rationales for therapeutic apheresis, Haematology, 10, 203-207.
15. G. Wehrli, 2005, Update on therapeutic apheresis, Current Hematology reports, 4: 477-482.
16. US Patent Publication No. 2003/0211471.
17. U.S. Pat. No. 4,011,067.
18. U.S. Pat. No. 4,604,203.
19. U.S. Pat. No. 5,328,758.
20. US Patent Publication No. 2006/0024727.
21. R. Klingel, P. Mausfeld, C. Fassbender and B. Goehlen 2004, Lipid filtration-safe and effective methodology to perform lipid-apheresis, Transfusion and Apheresis Science, 30, 245-254.
22. J. Svitel, A. Balbo, R. A. Mariuzza, N. R. Gonzales and P. Schuck 2003, Combined affinity and rate constant distributions of ligand populations from experimental surface binding kinetics and equilibria, Biophysical J., 84, 4062-4077.
23. R. A. Houghten 2000, Parallel array and mixture-based synthetic combinatorial chemistry: Tools for the next millennium, Ann. Rev. Pharmacology & Toxicology, 40, 273-282.
24. M. B. Pepys, G. M. Hirschfield, G. A. Tennent, et al. 2006, Targeting C-reactive protein for the treatment of cardiovascular disease, Nature 440, 1217-1221.
25. D. B. Kaufmnan, M. E. Hentsch, G. A. Baumbach, J. A. Buettner, C. A. Dadd, P. Y. Huang, D. J. Hammond and R. G. Carbonell 2002. Affinity purification of fibrinogen using a ligand from a peptide library, Biotechnology and Bioengineering 77, 278-289.
26. Á. Furka, F. Sebestyén, M. Asgedom, and G. Dibó 1991, General method for rapid synthesis of multicomponent peptide mixtures, International Journal Peptide Protein Research, 37, 487-493.
27. K. S. Lam, S. E. Salmon, E. M. Hersch, V. J. Hruby, W. M. Kaznierski, and R. J. Knapp (1991), A new type of synthetic peptide library for identifying ligand-binding activity, Nature 354, 82-84.
28. J. T. Lathrop, I. Fijalkowska, and D. J. Hammond 2007, The Bead Blot: A method for identifying ligand-protein and protein-protein interactions using combinatorial libraries of peptide ligands, Anal. Biochem. 361, 65-76.

What is claimed is:

1. An apheresis method for treating a subject with a condition of sustained elevation of CRP, comprising
(A) providing a support upon which is immobilized a ligand that has high affinity and high specificity for CRP, such that a CRP binding element is formed, and
(B) bringing said element into ex vivo contact with body fluid from said subject, whereby the CRP concentration in said subject is reduced.

2. The method of claim 1, wherein said support comprises a polysaccharide.

3. The method of claim 1, wherein said support comprises a synthetic polymer.

4. The method of claim 1, wherein said support is in the form of a membrane.

5. The method of claim 1, wherein the support is in the form of a resin.

6. The method of claim 1, wherein said ligand is immobilized on said support using a bifunctional linker.

7. The method of claim 1, wherein said ligand does not activate platelets, factor VII, complement, or angiotensin-converting enzyme.

8. The method of claim 1, wherein said ligand is a peptide from 3 to 25 amino acids in length.

9. The method of claim 1, wherein said ligand is a peptide from 3 to 15 amino acids in length.

10. The method of claim 1, wherein said ligand does not contain methionine or cysteine.

11. The method of claim 1, wherein said ligand is a peptide comprising an amino acid sequence selected from the group consisting of SEQ ID NOS: 1-8.

12. The method of claim 11, wherein said ligand is a peptide comprising an amino acid sequence selected from the group consisting of SEQ ID NOS: 4, 6 and 7.

13. The method of claim 1, wherein the association constant between said CRP and said ligand is at least 10⁶ M.

14. The method of claim 1, wherein said CRP comprises bound CRP.

15. The method of claim 1, wherein said subject is a human.

16. The method of claim 1, wherein said body fluid is whole blood.

17. The method of claim 1, wherein said body fluid is plasma.

18. The method of claim 1, wherein said CRP concentration in said body fluid or subject is reduced to 2 mg/mL or less.

19. The method of claim 18, wherein said CRP concentration in said body fluid or subject is reduced to 1 mg/mL or less.

20. The method of claim 1, further comprising returning said body fluid to said subject.

21. The method of claim 1, further comprising removing bound CRP from the support.

22. The method of claim 1, further comprising reducing LDL.

23. The method of claim 1, wherein said reduction of LDL is achieved by administering to said subject a pharmaceutical composition effective at reducing LDL.

24. A system for CRP apheresis, comprising (A) a support upon which is immobilized a ligand that has high affinity and high specificity for CRP, such that a CRP binding element is formed, and (B) apparatus for bringing said support into contact ex vivo with bodily fluid from a subject, thereby to affect CRP level in said bodily fluid, and for returning said bodily fluid to said subject.

25. The system of claim 24, wherein said support is a polysaccharide.

26. The system of claim 24, wherein said support is a synthetic polymer.

27. The system of claim 24, wherein said support is in the form of a membrane.

28. The system of claim 24, wherein the support is in the form of a resin.

29. The system of claim 24, wherein said ligand is immobilized on said support using bifunctional linker.

30. The system of claim 24, wherein said ligand does not activate platelets, factor VII, complement, or angiotensin-converting enzyme.

31. The system of claim 24, wherein said ligand is a peptide 3 to 25 amino acids in length.

32. The system of claim 24, wherein said ligand is a peptide from 3 to 15 amino acids in length.

33. The system of claim 24, wherein said ligand does not contain methionine or cysteine.

34. The system of claim 24, wherein said ligand is a peptide comprising an amino acid sequence selected from the group consisting of SEQ ID NOS: 1-8.

35. The system of claim 34, wherein said ligand is a peptide comprising an amino acid sequence selected from the group consisting of SEQ ID NOS: 4, 6, and 7.

36. The system of claim 24, wherein the association constant between said CRP and said ligand is at least 10⁶ M.

37. The system of claim 24, wherein said CRP comprises bound CRP.

38. The system of claim 24, wherein said subject is a human.

39. The system of claim 24, wherein said body fluid is whole blood.

40. The system of claim 24, wherein said body fluid is plasma.

41. The system of claim 24, wherein said CRP concentration in said body fluid of subject is reduced to 2 mg/mL or less.

42. The system of claim 41, wherein said CRP concentration in said body fluid of subject is reduced to 1 mg/mL or less.

43. The method of claim 24, further comprising removing bound CRP from the support.

44. A ligand that has high affinity and high specificity for CRP comprising, a peptide, wherein the association constant between said CRP and said ligand is at least 10⁶M.

45. The ligand of claim 44, wherein said peptide is from 3 to 25 amino acids in length.

46. The ligand of claim 45, wherein said peptide is from 3 to 15 amino acids in length.

47. The ligand of claim 44, wherein said peptide comprising an amino acid sequence selected from the group consisting of SEQ ID NOS: 1-8.

48. The ligand of claim 47, wherein said peptide comprising an amino acid sequence selected from the group consisting of SEQ ID NOS: 4, 6, and 7.

49. The ligand of claim 44, wherein said ligand does not activate platelets, factor VII, complement, or angiotensin-converting enzyme.

50. The ligand of claim 44, wherein said peptide does not contain methionine or cysteine.

51. The ligand of claim 44, wherein said peptide does not contain methionine, cysteine, or glutamine at the N-terminal position.

52. The ligand of claim 44, wherein said peptide is selected from a library.

53. The ligand of claim 44, further comprising a spacer.

54. The ligand of claim 52, wherein said spacer is β-alanine or ε-amino caproic acid.

55. The ligand of claim 52, wherein said spacer is a polyethylene oxide polymer.

56. The ligand of claim 44, wherein said CRP comprises bound CRP.
